(19) United States
(12) Patent Application Publication
(10) Pub. No.: US 2005/0041209 A1
(43) Pub. Date: Feb. 24, 2005

(54) Pupil position measuring apparatus and method

(76) Inventors: Oded Katzman, Haifa (IL); Dani Katzman, Haifa (IL); Youval Katzman, Zichron Yaacov (IL); Zohar Katzman, Haifa (IL)

Correspondence Address: NATH & ASSOCIATES, 1030 15th STREET, NW, 6TH FLOOR, WASHINGTON, DC, 20005, US

(21) Appl. No.: 10/633,303

(22) Filed: Aug. 4, 2003

(51) Int. Cl. A61B 3/10

(52) U.S. Cl. 351/204

(57) ABSTRACT

A method and a system for measuring an individual's pupil position (PP) in relation to a particular eyeglasses frame, wherein a display module (DM) is attached to the eyeglass's frame and a graphic image displayed on the DM is displaced until it coincides with the individual's line of vision, whereby the coordinates of the coincidence location are registered to obtain the PP.

### FIELD OF THE INVENTION

The present invention relates to ophthalmic equipment for eye-testing and spectacle setting. More specifically the invention is concerned with an apparatus and a method for accurately determining the position of the pupils of an individual and for correctly positioning lenses with respect to a spectacle frame of the individual.

### BACKGROUND OF THE INVENTION

When an individual requires corrective eyeglasses, it is extremely important that the corrective lenses be positioned in proper relationship to the individual's line of vision (in alignment with the individual's pupils and a viewed object). In particular this is the case when considering bifocal or multifocal lenses (also referred to as progressive lenses). However, this information is useful also for diagnostic and research purposes.

In order to obtain correct measurements, it is important that such measurements be carried out while the individual is wearing the eyeglasses at a normal and comfortable position (corresponding with how the wearer will use the eyeglasses), such that the relative positioning of the lenses with respect to the individual's eyes is most accurate. This measurement problem is further compounded by the fact that present day eyeglass frames take a wide variety of shapes and sizes ranging from generally circular in shape to various oval configurations. Additionally, any such measuring apparatus should also be capable of accommodating the so-called frameless eyeglasses as well as wire rim frames, etc. Even more so, it is important that the lenses be fitted in the frame in correspondence with the individual's regular activities, i.e. driving, reading, computer use, TV watching, outdoor activities, etc. and that measurements be taken at different positions i.e. leaning over, walking, sitting, etc.

A variety of measuring techniques and devices were proposed throughout the years, some of which are simple but not reliable and non accurate, e.g. when using manual measuring techniques. Other techniques are complicated to use thus requiring substantial amounts of time in obtaining the desired measurement. Others of these techniques and devices do not tend to be sufficiently accurate or may result in movement of the eyeglass frames during a critical portion, of the measuring process. Still other devices may be well suited for use with a particularly shaped eyeglass frame but may be unsuitable with respect to other shapes or types of frames such as the so-called frameless/rimless eyeglasses, wire-rim frames, etc.

U.S. Pat. No. 4,653,192 to Conard et al. discloses an apparatus for use in accurately determining the vertical position for the line of demarcation between the bifocal correction lens and other corrective lens segments to be provided in eyeglass lenses. The measuring apparatus includes an elongated indicia carrying member having provided thereon adjustable clamping members specifically designed to enable the elongated member to be attached to a wide variety of eyeglass frames for purposes of obtaining a measurement of the proper height for location of the segment line. The elongated member also includes a sliding gauge which may be moved there-along and cooperates with the indicia to provide an accurate, easily determined measurement for the position of the segment line.

U.S. Pat. No. 4,494,837 discloses a pupil location gauge having an index member movable in relation to a graduated scale for use in an ophthalmic test lens holder for subjective measurement of both vertical and horizontal pupillary distance. Either monocular or binocular measurements of the pupil location may be made with respect to a spectacle frame.

It is an object of the present invention to provide an apparatus and a method for carrying out ophthalmic measurements so as to obtain correct measurements indicating the position of an individual's pupils and to indicate correct positioning of lenses in proper relationship to the individual's line of vision with respect to a particular eyeglass of the individual.

### SUMMARY OF THE INVENTION

According to the present invention there is provided a method for measuring an individual's pupil position (PP) in relation to a particular eyeglasses frame, wherein a display module (DM) is attached to the eyeglass's frame and a graphic image displayed on the DM is displaced until it extends in alignment with the individual's pupils, whereby the coordinates of the alignment location are registered to obtain the PP.

The above method comprises many embodiments, for example:
The display module may be attached to the eyeglass's frame at a recognizable relation, or the relative position of the DM with respect to the eyeglass's frame may be determined through or after the process;
The graphic image may be displayed so as to interfere with the individual's line of vision while staring at a target mark;
The method may be carried out for determining the PP for both eyes simultaneously or separately for one eye at a time;
The graphic image may be part of a virtual image perceived upon blending of the graphic image with the target mark;
The graphic image may be part of a virtual image perceived upon blending of a graphic images simultaneously displayed in front of each eye.

The present invention is also concerned with a system for measuring an individual's pupil position (PP) in relation to a particular eyeglasses frame, the system comprising a fixture member for attaching to the eyeglasses frame at a recognizable relation, at least one display module (DM) suited for displaying a graphic image on the DM, a control unit for controlling and displacing the graphic image, and a register for picking up the coordinates of the graphic image location at selected positions.

Once the coordinates of the PP are registered they may be used for different purposes and in various ways. When the information is needed for shaping lenses so as to fit within the particular eyeglass of the individual, e.g. when fitting bi-focal or multi-focal lenses, the information may be directly applied to raw lenses fitted on the frame. Alternatively, the coordinates of the frame are obtained and the coordinates of the PP are superimposed (during or after the process) so the raw lenses may then be machined according to this information.

### BRIEF DESCRIPTION OF THE DRAWINGS

In order to understand the invention and to see how it may be carried out in practice, some embodiments will now be described, by way of non-limiting examples only, with reference to the accompanying drawings, in which:

FIGS. 1 schematically illustrates a system in accordance with the present invention, wherein:

FIG. 1A is a front view of an individual wearing eyeglass and fitted with a system according to the invention;

FIG. 1B is a side view of FIG. 1A;

FIG. 2A illustrates a system according to an embodiment of the present invention;

FIG. 2B is a local section of the portion marked I in FIG. 2;

FIG. 2C is an isometric representation of the portion marked II in FIG. 2;

FIGS. 3A to 3C illustrate several options of a display module according to the present invention;

FIG. 4A illustrates a system according to another embodiment of the present invention, at a first position;

FIG. 4B illustrates the embodiment of FIG. 4A at a second position.

### DETAILED DESCRIPTION OF THE INVENTION

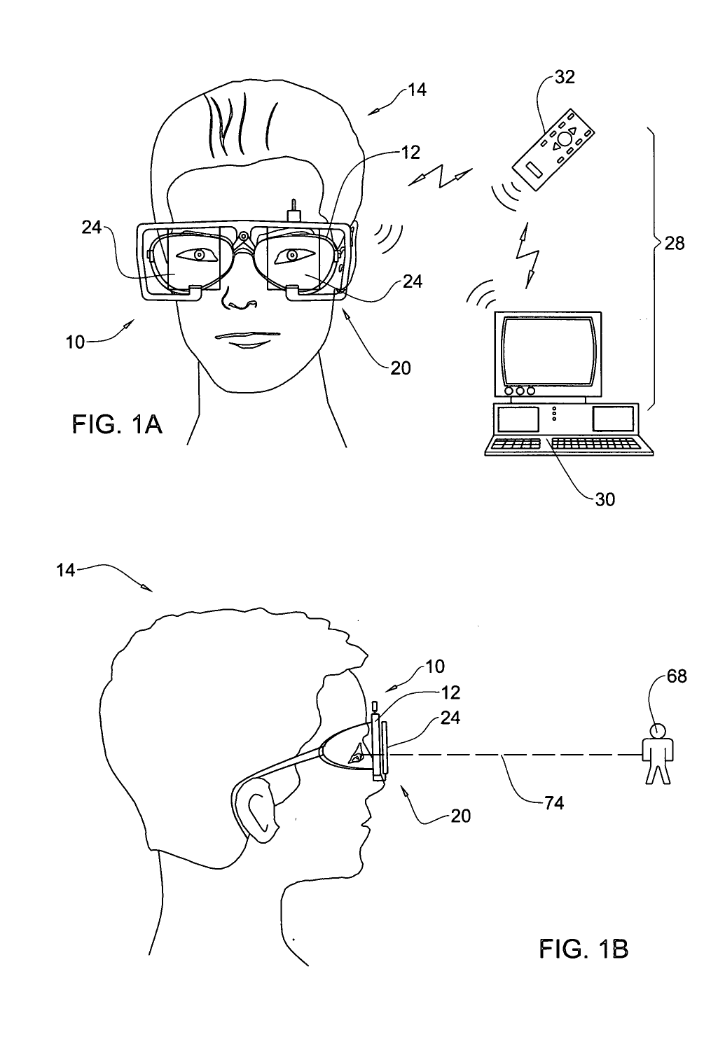

Attention is first directed to FIGS. 1A and 1B of the drawings illustrating a system in accordance with the present invention generally designated 10 mounted on a pair of spectacles (eyeglass) 12 of individual 14.

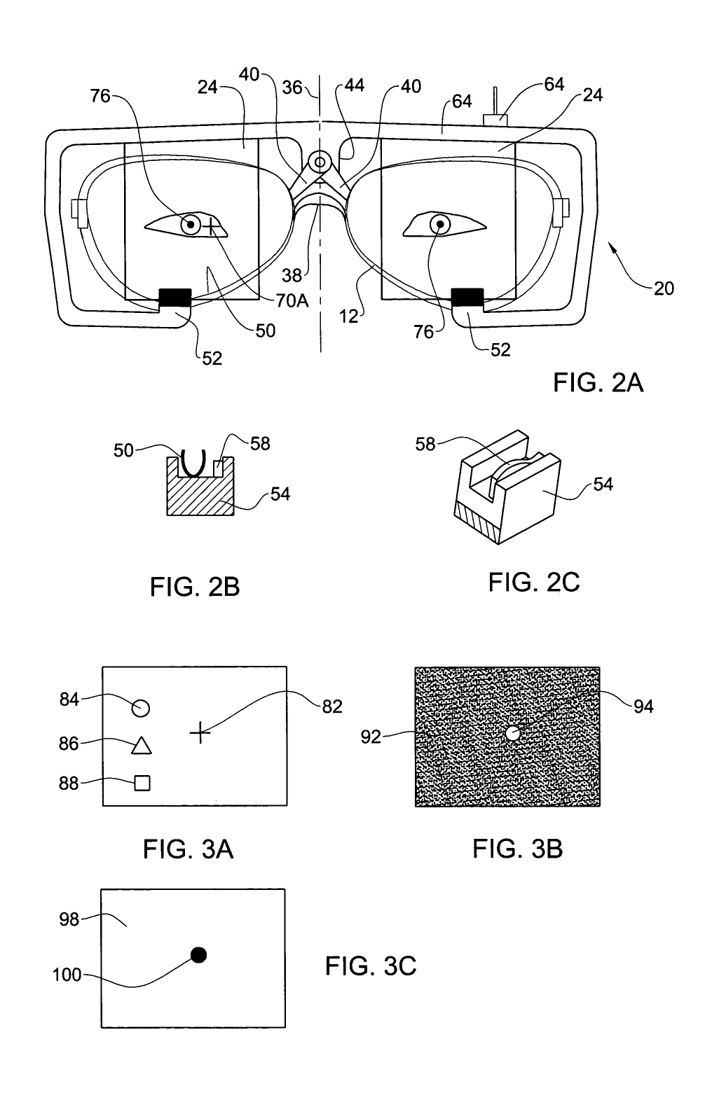

The system 12 comprises a fixture member generally designated 20 which will be discussed in further detail with reference to FIGS. 2. The fixture member of the embodiment seen in FIGS. 1A and 1B supports a pair of display modules (DM) 24 which at use extends opposite the individual's eyes.

The system further comprises a control assembly 28 (FIG. 1A) which in the present embodiment comprises a processor 30 and a controller 32 which in the present embodiment is a hand-held remote control device transmitting and receiving data to and from the device 20 and the controller 32. It is however appreciated that communications may be by a variety of different means, i.e. wire communication, IR communication, RF communication, etc. and further, that the controller 32 may be fitted with digital controls or mechanical controls.

In general, the fixture member 20 is of lightweight and does not interfere with wearing the eyeglass 12 whereby the individual 14 is comfortable during the measurement process.

With further reference being made to FIGS. 2A-2C, fixture member 20 has a frame-like structure fitted at a top center portion thereof with a nose-bridge engaging mechanism 36 fitted for self-centering about the bridge 38 of the eyeglass frame 12. The mechanism 36 comprises a pair of arms 40 pivotally mounted at 42 to an extension 44 of the frame-like portion 46. The arms 40 are spring biased so as to clampingly engage over the nose bridge 38 of the eyeglass and to self-center thereabout. Arms 40 bias the spectacle frame 12 downwards whereby a lower portion of the spectacle frame 50 (or of a raw spectacle lens in case of rimless eyeglass) engages into a receptacle portion 52 of the frame 46 of the device 20 and is securely nested by means of a leaf spring 58 so as to eliminate or reduce slack between the spectacle frame 12 and the frame 46 of device 20.

Display modules 24 are fixed to the frame 46, symmetrically positioned. The display modules (DM) may be a variety of display screens, e.g. an LCD screen or any other suitable graphic display module capable of displaying various patterns and images as well as changing its transparency through the range of transparent to opaque, as will be explained hereinafter in more detail with reference to FIGS. 3A-3C.

According to some particular embodiments, the graphic image may be part of a pattern or scheme which when blended with another graphic image (i.e. displayed for another second eye) is perceived as a virtual image. For example, the graphic image may be a first colored pattern (for example a blue circle) displayed in front of one eye and a second colored pattern (for example a yellow circle) displayed in front of the other eye, where only when both graphic images are in alignment with the respective pupils, a third color is perceived (a green circle in the particular example). The same result may be achieved by using other figures, e.g. geometric shapes. For example, if inverted half circles are displayed in front of each eye, the resultant perceived image, when both half circles are in alignment with the respective pupils, when both graphic images are in alignment with the respective pupils will be a complete circle.

A person versed in the art will appreciate that the image may be a reality image or a virtual image.

Similarly, if the target image constitutes part of the virtual image and the displayed graphic image (displayed on the DM) constitutes the other part of said virtual image, the resultant virtual image upon intersection of the graphic image with the line of vision, while staring at the target image, will be perceives as said virtual combined image. Accordingly, each eye may be presented with different images.

As already mentioned hereinabove, the process of locating and measuring the PP may be carried out by locating the point of intersection of the graphic image with the individual's line of vision while staring at a target mark (typically distant, from the individual) and also by displaying an image on a display monitor giving rise to a virtual image mimicking a distant image, and then locating the position of the individual's pupils when they are aligned and staring at the virtual image.

As an example, a display module (DM) according to an embodiment of the invention, is of the type known as a personal monitor (PM), where a high resolution color image is created in the individual's eyesight. The personal monitor is a monoscopic biocular display with a relatively narrow field of view, which can receive video signals from any video source. The signals are converted in the controller unit into signals driving the electronics of the LCD displays. The PM takes standard signals and displays them on a small TFT LCD display module that can be connected to any standard signal source e.g. video, computer, etc. The device comprises lenses and mirrors that project the displayed image into the retina of the eyes. The PM has a mounting slot that fits onto the nose piece of the eyeglasses.

However, other display modules are suitable for use with the present invention, e.g. so called reflective displays, etc.

The display module is typically a thin and lightweight surface which, in accordance with some particular embodiments, may have changing optical parameters to comply with near sighted or far sighted individuals (myopia/hyperopia) and further to adjust the brightness of the graphic image displayed on the module, depending on lightening conditions, etc. furthermore, it may be possible to adjust different optical parameters according to correcting tables, etc. in addition, some other optical elements may be used in conjunction with the apparatus of the invention. For example, power correcting lenses may be applied to correct sight deficiencies of the patient, filtering elements (e.g. to overcome glare, etc), prisms, etc.

The graphic image displayed on the display modules 24 is displaceable about the display module and is controllable by means of a controlling assembly (e.g. in FIG. 1A comprising processor 30 ad controller 32) which in the present embodiment is by wireless means and for that purpose a receiver/transmitter unit 64 is mounted on the frame 46.

In practical use, the device 20 in accordance with the present embodiment is securely fixed over the spectacle frame 12 and the eyeglass are then comfortably worn by the individual, as if the device 20 is absent. At this position, the coordinates of the spectacle frame 12 with respect to the frame 46 of device 20 are measured and registered by processor 30. Then, the individual is requested to stare at a target mark (68 in FIG. 1B; such a target mark may be a sign marked on a board or wall at a predetermined distance, etc.) and a graphic image is then displayed on the display module 24. The individual is requested to displace the graphic image (70A in FIG. 2A) until the graphic image interferes with the individual's line of vision 74 (FIG. 1B) while staring at the target mark 68.

The process may repeat several times (using the same graphic images or different ones at each time and optionally changing the size and distance of the target mark) and each time the graphic image intercepts with the individual's line of vision a point of interception is registered into the processor 30, e.g. by use of the control unit 32.

It is appreciated that the test may be carried out by the individual or by the professional. It is further appreciated that the measurements may be carried for one eye at a time while the other eye may be comfortably kept open and the display module in front of that eye may be darkened or made opaque to prevent sight interference. Alternatively, the measurement may be carried out for both eyes simultaneously.

At the end of the measurement process, processor 30 generates the coordinates of the pupils 76 with respect to the true position of the eyeglass frame 12, based on the processed data registered by the processor 30. The information may be processed by different statistic analysis as known per se with or without using correction factors to compensate and adjust for different parameters.

The arrangement may be such that during a measuring process the graphic image is constantly displayed and displaced, or, the graphic image may be displayed each time at a different position in a non-continues fashion.

The data concerning the position of the pupils 76 may be used in different ways and for different purposes such as, for example, for machining spectacle lenses according to optic prescriptions, research and study, etc.

It is appreciated that measurements of the pupils' position may be carried out for far distance and for near distance (i.e. reading position) which is advantageous in particular for manufacturing of multi focal lenses whereby indications are provided for determining the position of the individual's eyes during such positions.

With further reference now to FIGS. 3A-3C, there are illustrated several examples of display modules in accordance with the present invention. In FIG. 3A there is illustrated a display module 80 where the graphic image is in the form of a target mark 82. However, this target mark may have various shapes, e.g. circular, triangular, rectangular, etc., as exemplified in FIG. 3A and marked 84-88, respectively. In this example the display module 80 is substantially translucent and the graphic image is a fine image displayed on the screen to facilitate “aiming” by displacing the graphic image to intercept with the line of sight.

In the embodiment of FIG. 3B, the display module 92 is opaque whilst the graphic image 94 is a translucent image, in the present example being a circle. In this embodiment, the translucent portion 94 is displaced until the image appears through the translucent portion. In order to facilitate location of the target mark through the miniature graphic image 94, the background of the display module may gradually change from translucent to opaque. Alternatively, the translucent graphic image 94 may have a “keyhole” like shape where a larger landscape portion may be visioned though the opaque background to facilitate focusing of the target mark to the graphic image.

In the embodiment of FIG. 3C, the display module 98 is translucent whilst the graphic image 100 is a dark spot, circular in the present embodiment.

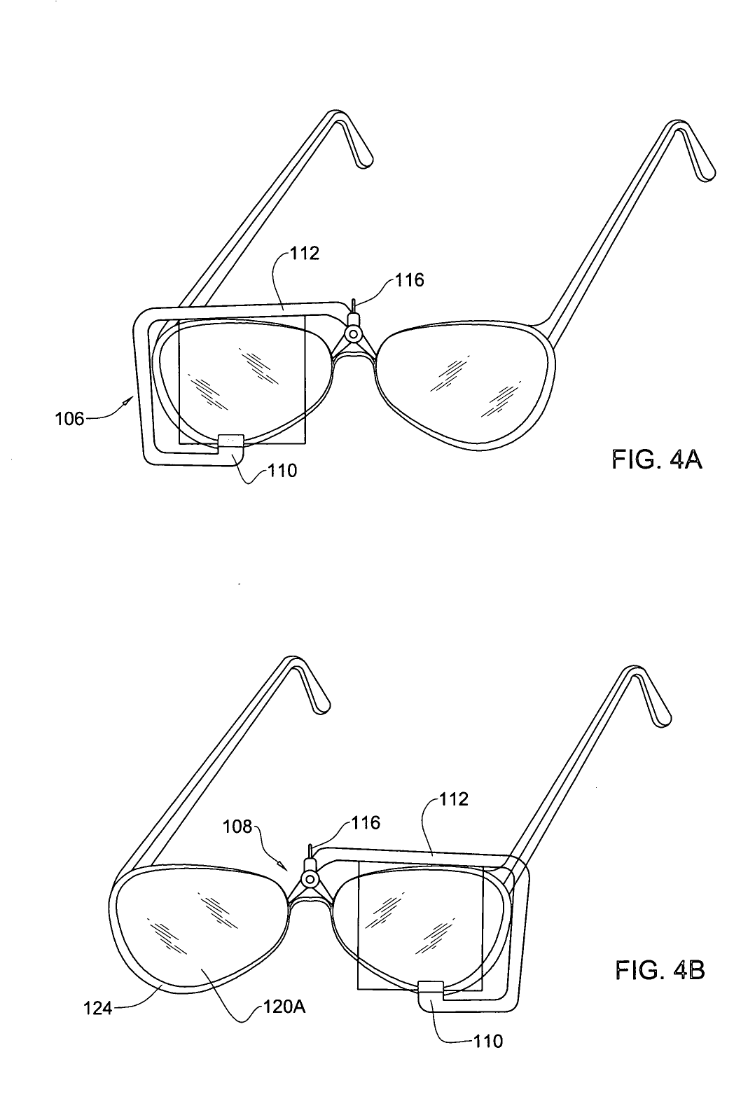

With further reference being made to FIGS. 4A and 4E, there is illustrated a modification of a device in accordance with the present invention generally designated 106 which is principally similar to the device 20 in FIG. 2A with the exception that the device 106 is a single module device comprising a self-centering clamping mechanism 108 similar to the arrangement of FIG. 2A and a similar engagement portion 110 at a lower part of the frame 112 whereas the frame 112 is either pivotal about an access 116 extending from mechanism 108 or mounted each time in front or behind a different lens 120A and 120B of the spectacle frame 124. Similarly, the DM may be mounted within the specific eyeglass frame.

It is appreciated that a variety of attaching mechanisms are available for securely attaching the frame of the device in accordance with the present invention to a variety of different eyeglass frames. Such a device may be attached to both raw lenses or to one lens at a time. It is further to be clear that the DM may by itself attachable to the eyeglass without the need for additional support frame. For example, the device may be attached to the spectacle frame by vacuum-suction cups attached to raw lenses of the eyeglass. Other attachment means may be, for example, magnet arrangements, clips or snap-on arrangements, etc.

Whilst some embodiments have been, described and illustrated with reference to some drawings, the artisan will appreciate that many variations are possible which do not depart from the general scope of the invention, mutatis, mutandis.

1. A method for measuring an individual's pupil position (PP) in relation to a particular eyeglasses frame, the method comprising the following steps:
(i) fitting a display module (DM) to the eyeglass's frame;
(ii) displaying on the DM a graphic image and displacing it until it interferes with the individual's line of vision; and
(iii) registering the coordinates of the interference location to obtain the PP.

2. A method according to claim 1, wherein the display module is fitted to the eyeglass's frame at a recognizable location.

3. A method according to claim 1, wherein the graphic image is displayed while the individual is staring at a target mark.

4. A method according to claim 1, wherein the display module comprises an LCD screen.

5. A method according to claim 3, wherein the graphic image is a transparent image created on a screen surface with clarity thereof changeable between transparent to opaque, wherein the target mark is viewed through the transparent image area.

6. A method according to claim 3, wherein the graphic image is a fine aiming image created on the DM, where the target image is substantially not bidden by said graphic image upon interference with the individual's line of vision.

7. A method according to claim 3, wherein the graphic image is a dark image formed on a substantially clear surface of the DM, where the target image is substantially hidden by said graphic image upon interference with the individual's line of vision.

8. A method according to claim. 1, wherein controlling the graphic image and its displacement is carried out by the individual.

9. A method according to claim 1, wherein controlling the graphic image and its displacement is carried out by a trained personnel.

10. A method according to claim 1, wherein the PP is separately detected and measured for each eye.

11. A method according to claim 1, wherein the display module is mounted on a fixture member fixable to the eyeglass's frame.

12. A method according to claim 1, wherein the display module (DM) is positioned behind or in front of the eyeglass's frame.

13. A method according to claim 1, wherein the display module (DM) replaces the eyeglass's lenses.

14. A method according to claim 1, carried out with or without raw lenses fitted on the eyeglass's frame.

15. A method according to claim 1, wherein steps (b) and (c) are repeated for target marks at a range of distances and positions.

16. A method according to claim 1, wherein optical parameters and factors are compensated by a conventional optics or by a electronic display controlled by a processor unit, to correspond with actual imaging factors.

17. A method according to claim 16, wherein the factors comprise distance, illumination, lens power, myopia/hyperopia, glare, brightness.

18. A method according to claim 1, wherein the displayed image is a reality image or a virtual image.

19. A method for measuring an individual's pupil position (PP) for positioning a fitting point (FP) of a lens such that it extends in proper relationship to the individual's line of vision with respect to particular eyeglass, the method comprising the following steps:
(i) fitting a display module (DM) in fixed and recognizable relation to the eyeglass's frame;
(ii) displaying on the DM a graphic image and displacing it until it aligns with the individual's pupil position;
(iii) registering the coordinates of the alignment position to obtain the PP; and
(iv) converting the alignment position coordinates to align the FP of the lens with respect to the individual's PP.

20. A method according to claim 18, wherein the graphic image is displaced until it intersects with the individual's line of vision, while staring at a target mark.

21. A method for measuring an individual's pupil position (PP) in relation to a particular eyeglass's frame, wherein a display module (DM) is fixed in recognizable relation to the eyeglass's frame and a graphic image displayed on the DM is displaced until it interferes with the individual's line of vision while staring at a target mark, whereby the coordinates of the interference location are registered to obtain the PP.

22. A system for measuring an individual's pupil position (PP) in relation to a particular eyeglasses frame, the system comprising a fixture member for attaching to the eyeglasses frame, at least one display module (DM) suited for displaying a graphic image on the DM, a control unit for controlling and displacing the graphic image, and a register for picking up selected location coordinates of the graphic image.

23. A system according to claim 22, wherein the fixture member is attached to the eyeglass's frame at a recognizable relation.

24. A system according to claim 22, further comprising a controllable processor and a user interface for controlling the type and position of the graphic image and for registering the coordinates of the interference location.

25. A system according to claim 23, wherein the user interface is wireless unit.

26. A system according to claim 22, wherein the display module comprises an LCD screen.

27. A system according to claim 22, wherein the display module is controllable to change its clarity through the range of transparent to opaque.

28. A system according to claim 26, wherein the graphic image is a fine aiming image created on the DM, where a target image is substantially not hidden by said graphic image upon interference with the individual's line of vision.

29. A system according to claim 22, wherein the graphic image is a dark image formed on a substantially clear surface of the DM, where a target image is hidden by said graphic image upon interference with the individual's line of vision.

30. A system according to claim 22, wherein the display module is mounted on a fixture member fixable to the eyeglass's frame and comprising a centering mechanism.

31. A system according to claim 22, wherein the fixture member comprises a self centering mechanism so as to spontaneously center with respect to the eyeglass's frame.

32. A system according to claim 22, wherein the fixture member bears on the eyeglass's frame at a nose-bridge portion thereof.

33. A system according to claim 31, wherein the fixture member bears on the eyeglass's frame at least at another location to obtain true-position coordinates of the fixture versus the eyeglass's frame.

34. A system according to claim 22, wherein the DM is attached to the eyeglass's frame by a vacuum attachment cup applied to raw lenses of the eyeglass.

35. A system according to claim 22, comprising a single DM and where the fixture member is fixable to the eyeglass's frame and shiftable between a first position where the DM corresponds with a first lens of the eyeglass and a second position corresponding with a second lens of the eyeglass.

36. A system according to claim 22, wherein the fixture member supports a single display module shiftable between two positions corresponding with two lenses of the eyeglass's frame.

37. A system according to claim, 22, wherein the display module (DM) is positioned behind or in front of the eyeglass's lenses.

38. A system according to claim 22, carried out with or without raw lenses fitted on the eyeglass's frame.

39. A system according to claim 22, wherein an optical power of the display module is changeable to comply with individual's near/far sight.

40. A system according to claim. 22, wherein the graphic display is created on a reflective DM.

41. A system according to claim 22, wherein the graphic image is part of a pattern which when blended with another graphic image is perceived as a virtual image.

42. A system according to claim 41, wherein the graphic image is a colored pattern, wherein upon alignment of the graphic image with the individual's line of sight, a different colored pattern is perceived.

43. A system according to claim 41, wherein a different graphic image is displayed to each eye, wherein upon alignment of the graphic images with the individual's pupils a resultant perceived image appears, which is an image blending the two graphic images.

44. A system according to claim 22, wherein the displayed image is a reality image or a virtual image.
